(19) United States
(12) Patent Application Publication
(10) Pub. No.: US 2023/0307091 A1
(43) Pub. Date: Sep. 28, 2023

(54) PROGRAMMATIC PROCESSING OF PROTEIN OR NUCLEIC ACID SEQUENCES TO IDENTIFY MUTATIONS AT PROGRAMMATICALLY DETERMINED SUBSEQUENCES

(71) Applicant: Waters Technologies Ireland Limited, Dublin 2 (IE)

(72) Inventors: Scott J. Geromanos, Middletown, NJ (US); Emmy Maria Hoyes, Richmond (GB); Francis Tracey, Jericho, VT (US); Allen Caswell, Franklin, MA (US); Ming Gao, Milford, MA (US)

(21) Appl. No.: 18/187,792

(22) Filed: Mar. 22, 2023

Related U.S. Application Data
(60) Provisional application No. 63/322,506, filed on Mar. 22, 2022.

(51) Int. Cl. G16B 20/20; G16B 30/00

(52) U.S. Cl. CPC G16B 20/20; G16B 30/00

(57) ABSTRACT

The exemplary embodiments may obtain protein, gene sequence or nucleic acid sequences from data sources, process the sequences with a processor executing computer programming instructions to identify mutations and display information regarding the mutations to a user on a display device. For protein sequences, the exemplary embodiments may identify which subsequences in the sequences are well suited for observing as positions in the sequences where possible mutations may arise. Statistical techniques may be applied to variations in the subsequences to determine whether the variations are mutations or not. The displayed information regarding each mutation may include the nature of the mutation, the frequency of the mutation, the location of the user having the mutation, the date that the sample was obtained and other information of interest.

### RELATED APPLICATIONS

This application claims the benefit of U.S. Provisional Patent Application No. 63/322,506, filed Mar. 22, 2022, the contents of which are incorporated herein by reference in their entirety.

### BACKGROUND

Proteins contain sequences of amino acids, and nucleic acids contain sequences of nucleotides. Mutations may be present in the nucleic acids and proteins. These mutations may pose challenges to diagnostic efforts that rely on detecting portions of the proteins or nucleic acids. For example, an assay that looks for certain amino acid sequences in the peptides may not work properly if one or more of the peptides have mutated as the amino acid sequences may have changed as a result of the mutation. The mutations also may pose a challenge to therapeutic measures like vaccines. The vaccines for viruses may be formulated to target a virus with a particular genetic fingerprint. When the genetic fingerprint changes, the vaccines may not be as effective.

### SUMMARY

In accordance with a first inventive aspect, a method is performed by a processor of an electronic device. The method includes programmatically determining with the processor that a selected subsequence for a protein, gene sequence, or nucleic acid is well suited for identifying mutations in gathered sequences of the protein, gene sequence, or nucleic acid relative to a reference sequence. The method also includes analyzing the gathered sequences with the processor to identify any variations in the selected subsequence, where the selected subsequence in the gathered sequences is different from the selected subsequence in the reference sequence. For ones of the gathered sequences where one or more variations in the selected subsequence have been identified, the processor determines whether the variation constitutes a mutation or not by applying a statistical test based on the frequency of the variation across the gathered sequences. The frequencies of mutations in the gathered sequences are determined, and information is output regarding the frequencies of the mutations in the gathered sequences.

The step of programmatically determining with the processor that a selected subsequence for a protein, gene sequence, or nucleic acid is well suited for identifying mutations in gathered sequences of the protein, gene sequence, or nucleic acid relative to a reference sequence may include (1) designating subsequences that are specific to the protein or to the nucleic acid, (2) designating subsequences in the protein, gene sequence, or nucleic acid that ionizes well, and/or (3) designating subsequences in the protein, gene sequence, or nucleic acid that exhibit good mass selectivity as candidates for being the at least one selected subsequence in the protein, gene sequence, or the nucleic acid that are well suited for identifying mutations in the gathered sequences relative to a reference sequence. In addition, the step of choosing one or more subsequences among the candidates to be the at least one selected subsequence that is well suited for identifying mutations in the gathered sequences relative to the reference sequence may be included.

The step of programmatically determining with the processor that at a selected subsequence for a protein, gene sequence, or nucleic acid is well suited for identifying mutations in gathered sequences of the protein, gene sequence or nucleic acid relative to a reference sequence may determine that multiple subsequences are well suited for identifying mutations in the gathered sequences relative to the reference sequence. The method may include programmatically, with the processor, retrieving the gathered sequences from a database. The method may include sequence aligning the gathered sequences with the processor. A statistical test may, based on a likelihood for each variation, determine whether the variation is a mutation. The outputting of the information regarding the frequencies of the mutations in the gathered sequences may include generating a web page, a file, or a user interface element for display that contains the information regarding the frequencies of the mutations in the gathered sequences. The outputting may also output additional other or additional information, such as metadata. The outputting of the information regarding the frequencies of the mutations in the gathered sequences may include outputting graphics depicting the frequencies of the mutations in the gathered sequences. The outputting of the information regarding the frequencies of the mutations in the gathered sequences may output the frequencies sorted by location where the gathered sequences were gathered and/or the dates when the gathered sequences were gathered, what each of mutations is and/or a frequency of each of the mutations. The protein may be one of a protein found in a virus, disease or disorder.

Computer programming instructions that cause the processor to perform the method when executed by a processor may be stored on a non-transitory computer-readable storage media.

In accordance with another inventive aspect, an electronic device includes a storage for storing computer programming instructions and a processor configured to execute the computer programming instructions. The computer programming instructions when executed cause the processor to programmatically determine that a selected subsequence is well suited for identifying mutations in the gathered sequences of a protein or nucleic acid relative to a reference sequence and analyze the gathered sequences to identify variations where the selected subsequence in the gathered sequences is different from the selected subsequence in the reference sequence. For ones of the gathered sequences where the selected subsequence is identified as different from the subsequence in the reference sequence, the computer programming instructions when executed by the processor cause the processor to determine whether there is a mutation or a non-mutation variation by applying a statistical test, to determine frequencies of mutations in the gathered sequences, and to output information regarding the frequencies of the mutations in the gathered sequences.

The outputting of the information regarding the frequencies of the mutations in the gathered sequences may include generating a web page, a file, or a user interface element for display that contains the information regarding the frequencies of the mutations in the gathered sequences.

### BRIEF DESCRIPTION OF THE DRAWINGS

FIG. 1A depicts different types of sequences that may be processes in exemplary embodiments.

FIG. 1B depicts an illustrative portion of a protein sequence and an illustrative portion of a nucleic acid sequence.

FIG. 1C depicts a flowchart of illustrative steps that may be performed in exemplary embodiments in processing sequences.

FIG. 2A depicts a flowchart of illustrative steps that may be performed in exemplary embodiments in identifying subsequences of interest.

FIG. 2B depicts a flowchart of illustrative steps that may be performed in exemplary embodiments in processing a subsequence to determine whether it is a subsequence of interest.

FIG. 2C depicts fields that may be included in a local database in exemplary embodiments.

FIG. 3 depicts a flowchart of illustrative steps that may be performed in exemplary embodiments in determining whether a variation is a mutation or not.

FIG. 4 depicts an illustrative table that may be output on a display device in exemplary embodiments to identify mutations and their frequencies.

FIG. 5 depicts a flowchart of illustrative steps that may be performed in exemplary embodiments in processing a test sample to determine if a patient has a virus and to identify any virus mutations.

FIG. 6 depicts a flowchart of illustrative steps that may be performed in exemplary embodiments to determine if a patient has cancer.

FIG. 7 depicts an illustrative networked computing environment suitable for exemplary embodiments.

FIG. 8 depicts a block diagram of an illustrative server/cluster for exemplary embodiments.

FIG. 9 depicts a block diagram of an illustrative client computing device for exemplary embodiments.

FIG. 10 depicts processing of a sample in accordance with exemplary embodiments.

### DETAILED DESCRIPTION

The exemplary embodiments may obtain protein, gene, or nucleic acid sequences from data sources, process the sequences with a processor executing computer programming instructions to identify mutations and display information regarding the mutations to a user on a display device. For protein sequences, the exemplary embodiments may identify which subsequences in the sequences are well suited for observing as positions in the sequences where possible mutations may arise. Statistical techniques may be applied to variations in the subsequences to determine whether the variations are mutations or not. The displayed information regarding each mutation may include the nature of the mutation, the frequency of the mutation, the location of the user having the mutation, the date that the sample was obtained and other information of interest. The displayed information also may include metadata regarding a patient, such as gender, age, race, weight or the like. The displayed information may include other information with a sample.

In some exemplary embodiments, the protein may be part of a virus, such as the influenza virus or the SARS-CoV2 virus. In other instances, for example, the protein may be indicative of a disease or disorder. The exemplary embodiments may gather sequences for users that have contracted the viruses and identify mutations. Thus, the computer programming instructions of the exemplary embodiments may help track mutations in viruses and identify both their geographic location and frequency. This may be useful in monitoring the behavior and spread of the viruses.

The exemplary embodiments are not limited in application to sequences for viruses but more generally may be applied to other types of nucleic acid and protein sequences. One application is for protein or genetic material found in a tissue of a patient. For this application, the processing may be able to identify mutations associated with disease or other abnormalities found in the tissue.

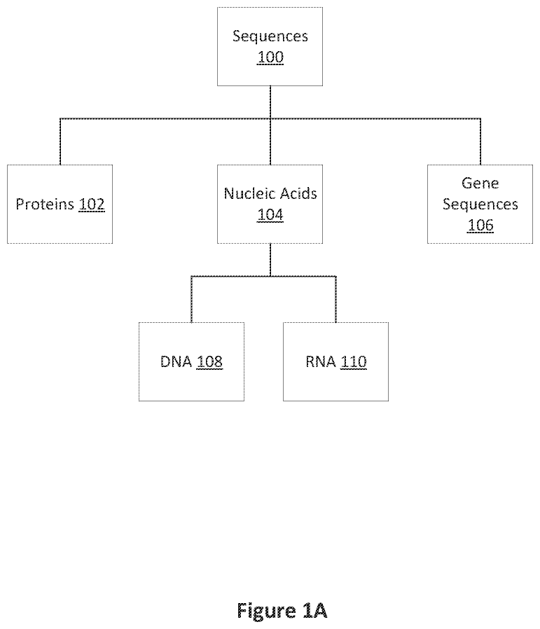

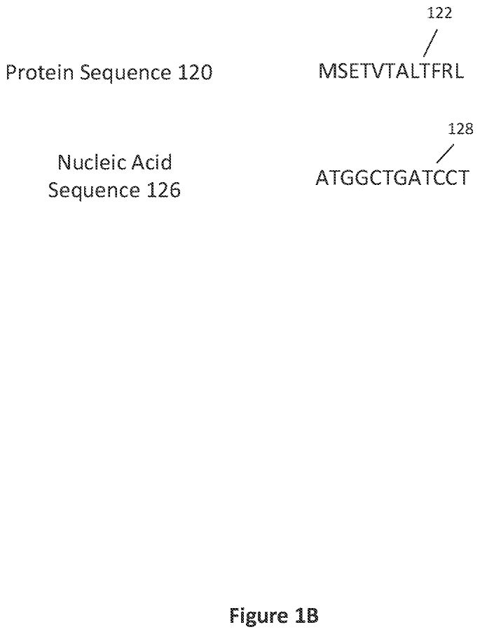

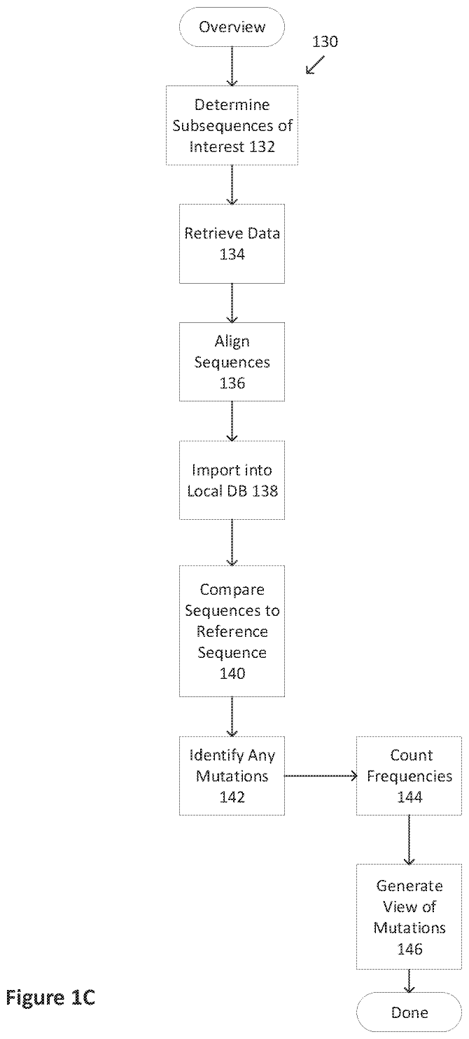

The exemplary embodiments are applicable to different types of sequences of biological information. As shown in FIG. 1A, the sequences 100 may include sequences of proteins 102 or sequences of nucleic acids 104 or other gene sequences 106. The protein sequences 102 are sequences of amino acids, whereas the nucleic acid sequences 104 are sequences of nucleotides. The nucleic acid sequences 104 may be for DNA 108 or RNA 110.

FIG. 1B depicts an example of a portion of a protein sequence 120 and a nucleic acid sequence 126. The protein sequence 120 contains a sequence of characters 122 where each character is associated with an amino acid. For example, the first character “M” in the sequence of characters 122 represents the amino acid methionine. The nucleic acid sequence 126 contains a sequence of characters 128 where each character represents a nucleotide. For example, the first character in the sequence of characters 128 represents the nucleotide adenine.

FIG. 1C depicts a flowchart 130 of illustrative steps that may be performed in exemplary embodiments regarding a protein sequence. Initially, at 132, the subsequences of interest in the sequences are determined by software executing on a processor of a computing device or other electronic device. The subsequences may be peptides when the sequence is a protein sequence. Each peptide is a chain of amino acids. The subsequences may be groups of sequential nucleotides constituting a portion of the sequences when the sequences are nucleic acid sequences. The subsequences of interest specify positions in the sequences where the processing looks for variations and determine whether the variations are mutations are not.

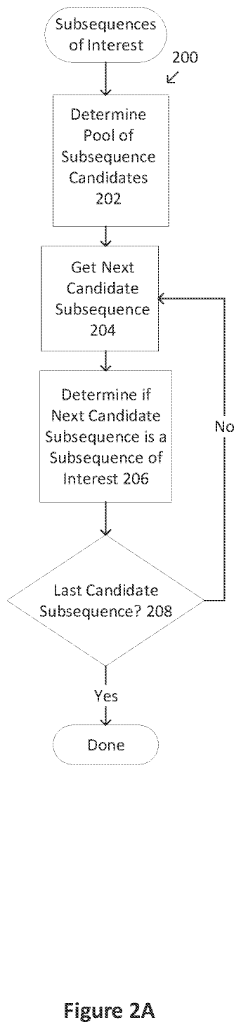

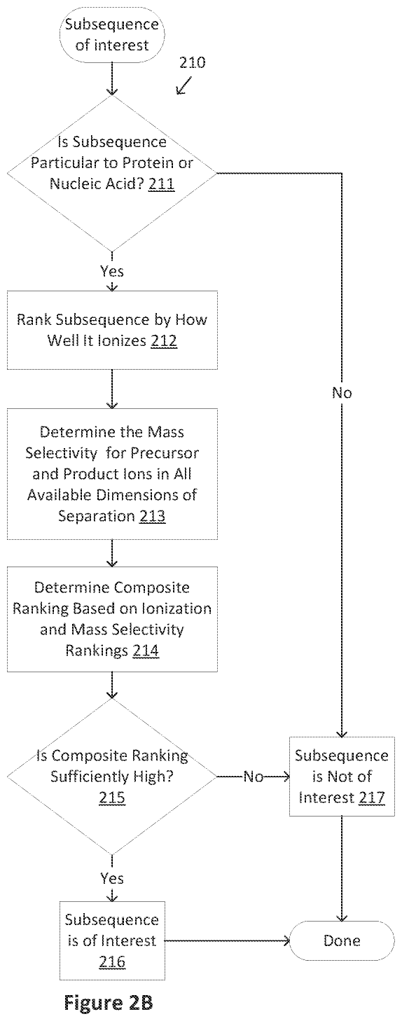

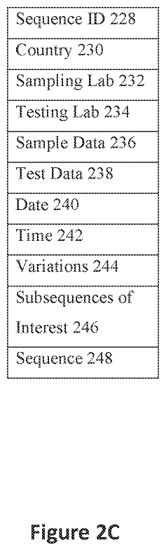

FIG. 2A depicts a flowchart 200 of illustrative step that may be performed in exemplary embodiments to identify subsequences of interest. At 202, the pool of subsequence candidates is determined. This may entail simply making each subsequence found in a reference sequence a candidate sequence or may instead entail identifying a subset of the subsequences in the reference sequence as candidates based on experience or other characteristics. The reference sequence may be a generally accepted sequence for the protein or nucleic acid. For example, statistical data may indicate that mutations primarily occur on 3 subsequences. As a result, those 3 subsequences may then be designated as the pool of subsequence candidates. At 204, a next subsequence in the pool of candidate subsequences is obtained for processing. At 206, a determination is made whether the next candidate subsequence is a subsequence of interest, as will be discussed in more detail below with reference to FIG. 2B. At 208, a check is made whether the candidate subsequence is the last subsequence in the pool of candidate subsequences. If so, processing is done. If not, the process repeats with a next candidate subsequence beginning at 204.

A variety of factors may be considered in determining whether a subsequence is a subsequence of interest. FIG. 2B depicts a flowchart 210 of illustrative steps that may be performed to determine whether a candidate subsequence in the sequence is a subsequence of interest in exemplary embodiments. The exemplary embodiments may automatically determine whether a candidate is well suited for being a subsequence of interest without the need for a user to identify the subsequence. These steps may be performed for each candidate subsequence to determine if the subsequence is one of interest. It should be appreciated that in some embodiments other criteria may be applied to determine whether to determine if a candidate subsequence is one of interest. For instance, the availability of an antibody for enrichment and the inability to resolve a candidate chromatographically are examples of other criteria. At 211, a determination is made to see if the candidate subsequence under consideration as a subsequence of interest is specific to the protein, gene sequence or nucleic acid. In other words, the processing measures how unique the subsequence is to the protein, gene sequence, or nucleic acid. Above a certain threshold level of uniqueness is acceptable. Otherwise, at 217, the subsequence is deemed to not be a subsequence of interest.

If the subsequence is sufficiently specific to the protein or nucleic acid, at 212, where the sequence is to be identified by applying a mass spectrometer (MS) or liquid chromatography mass spectrometer combination (LCMS), the subsequence is ranked by how well it ionizes relative to other subsequences. A subsequence that does not ionize well, will not be readily detectable by an MS or LC-MS system. If the subsequence does not ionize well, the subsequence is designated as not a subsequence of interest at 217. This step may be skipped where the elements of the sequence are identified using a different technique that does not involve MS.

At 213, the mass selectivity of both precursor ions and products ions for the subsequence across all dimensions of separation is determined and scored and/or ranked. Thus, the precursor ions and product ions for the subsequence are analyzed to determine their mass selectivity relative to the separation and detection and technologies that will be used, like mass spectrometry and chromatography, such as liquid chromatography. The mass spectrometer and the chromatography unit must be able to sufficiently mass resolve the precursor ions and product ions that result after ionization of the subsequence and mass spectrometry are applied. This step determines this ability to mass resolve for the subsequence.

At 214, a composite ranking for the subsequence is determined based on the ionization ranking and the mass selectivity for the subsequence. The composite ranking, for example, may be based on a weighted sum of the ionization ranking and a mass selectivity score or ranking. At 215, a check is made whether the composite ranking is sufficiently high or not. If the composite score is sufficiently high, the subsequence is determined to be a subsequence of interest at 216. If the composite score is not sufficiently high, the subsequence is determined to be not a subsequence of interest at 217.

It should be appreciated that the characteristics checked in FIG. 2B are intended to be illustrative and not limiting. Different characteristics than those checked in FIG. 2B may be checked to determine if a candidate subsequence is a subsequence of interest. Moreover, in some exemplary embodiments only a subset of the characteristics may be checked or alternatively, additional characteristics may be checked.

With reference to FIG. 1C once again, at 134, the sequence data is retrieved from a data source. The data source, may for example, be a public database that is accessible over the Internet or another networked connection. There are a variety of publicly accessible databases that hold protein and/or nucleic acid sequences for viruses, diseases, bacteria, and other pathogens. The data source, however, may be simply a storage location and may be located either remotely or locally. The sequence data may be retrieved from the databases using a scraper tool.

The sequences that have been retrieved need to be aligned because the sequences may not yet be aligned to a common reference sequence. Hence, at 136, the retrieved sequences are aligned. Aligning the sequences facilitates comparison of the sequence to a reference sequence to identify similarities and differences. The alignment of the sequences may be performed by a tool such as the bio.align package. The alignment may be performed relative to the reference protein sequence or nucleic acid sequence.

At 138, the aligned sequences are imported into a local database for processing. Each sequence may contain a header as well as sequence data. The importing may entail stripping metadata out of the header and storing the metadata in the local database along with the aligned sequences. One suitable format for the sequence data is the FASTA format. Since the format of the imported sequences is known, the fields in the header are known, and the header can be disassembled to extract information for storage in the local database.

Each sequence is one of a protein or genetic material found in a sample taken from a party for testing. For example, the testing may indicate whether a party has a virus or not. For testing for a disease or abnormality, such as for the SARS-CoV2 virus, a sample is obtained from a user. For the SARS-CoV2 virus, the sample likely would be a nasal swab. For other varieties of tests, saliva samples, urine samples, blood samples or other samples of bodily fluids or tissue may be obtained. The sample is processed by a sampling lab that determines the sequence for the protein or nucleic acid. The sequence may then be analyzed by a testing lab (which may be the same lab as the sampling lab in some instances) to determine whether the test is positive or negative.

FIG. 2C shows an example of some of the fields that may be stored in a local database for a particular sequence. This depiction is not intended to be an exhaustive listing of all possible fields that may be stored in the database. This depiction of the fields is intended to be merely illustrative and not limiting. The local database may store not only information extracted from the retrieved samples but also information resulting from local analysis of the sequence.

As shown in FIG. 2C, country information 230 regarding the country where the test sample was obtained may be stored in the local database. Sampling lab information 232 that identifies the sampling lab as well as testing lab information 234 that identifies the testing lab may be stored in the local database. Sample data 236 regarding the sample and test data 238 may be stored in the local database. Date information 240 and time information 242 of the sampling may be stored in the local database. Identified variations 244 in the subsequences of interest may be stored in the local database after the variations are identified. The identity of the subsequence of interest 246 may be stored in the local database. The sequence 248 may be stored in local database.

The local database may store the fields depicted in FIG. 2C in relational database tables. Alternatively, an object-oriented database may be used where an object is created for each sequence. Each sequence object may have attributes or data members for the depicted fields.

Once the sequences and associated metadata have been imported into local database (see 138 in FIG. 1C), the sequences may be processed by first comparing each sequence to the reference sequence at 140. The comparison looks at the positions of the subsequences of interest and identifies any variations between the reference sequence and the sequence being compared. All variations are noted.

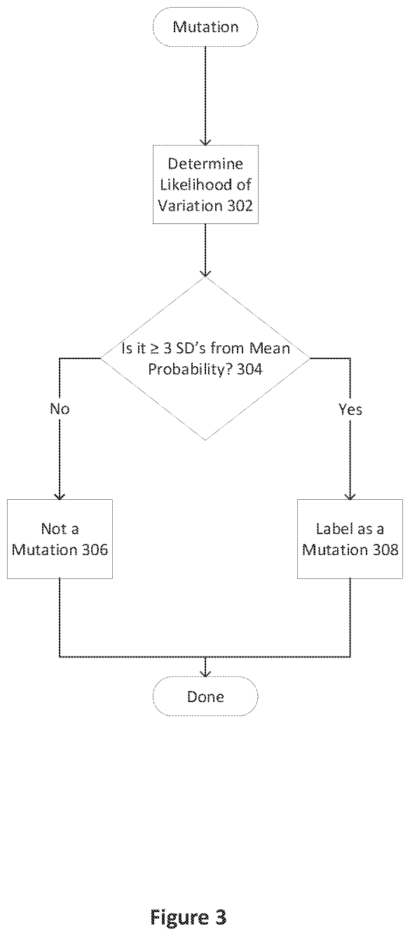

At 142, the variations are processed to identify any mutations. FIG. 3 depicts a flowchart of illustrative steps that may be performed in exemplary embodiments to identify the mutations. This identification of the mutations may be done by a processor executing computer programming instructions so that a user need not manually make the determination. At 302, the likelihood of a variation is determined. This likelihood may be calculated by processing empirical data for similar sequences to those that were retrieved to identify how often the variation occurred in the empirical data and then determining the likelihood. Alternatively, such likelihood information may already be available from previous analysis. Statistical analysis is then applied to determine whether the variation is within the expected variability for such sequences or lies outside the expected variability. Hence, at 304, a check is made as to whether then likelihood of the variation is 3 standard deviations or more outside the norm. Such variations are unlikely (approximately only 1% of all variations). If the variation has a likelihood that is 3 standard deviations or more from the norm, at 308, the variation is deemed a mutation. If not, at 306, the variation is deemed to not be a mutation.

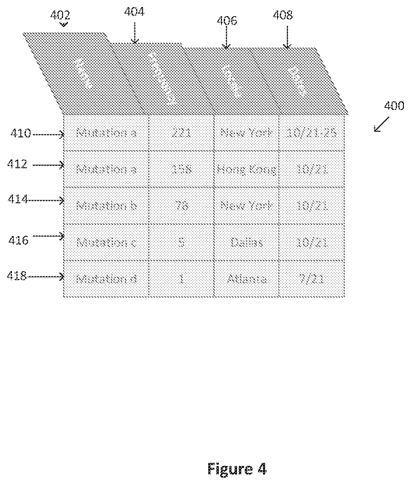

At 144, each variety of mutation is counted to determine the frequency of the mutation in the data set of sequences that have been retrieved and processed in the local database. At 146, a view of the results of the processing may be output. The output may be textual, tabular, graphical or a combination thereof. The output may be in the form of a web page, a user interface or even a file. FIG. 4 depicts an example of a possible tabular output. A table 400 is displays information regarding each variety of mutation. Column 402 holds the name of the mutation, column 406 holds the locale where the mutation appeared, column 408 hold the date or dates for the samples from which the sequences originated and column 404 specifies the frequency of the named mutation at the locale on those dates. For example, row 410 indicates that mutation a appeared 221 in data originating from New York for the dates of October 21 to October 25. Row 412 also concerns mutation a but holds data for Hong Kong on October 21. Row 414 specifies data for mutation b in New York on October 21, row 418 specifies data for mutation c in Dallas on October 21, and row 418 specifies data for mutation d in Atlanta on July 21.

The output information is helpful for tracking the frequency that a mutation has arisen. The output information helps to track mutations by locale and time window. This information can be helpful in tracking a mutation and understanding where the mutation is prevalent or not.

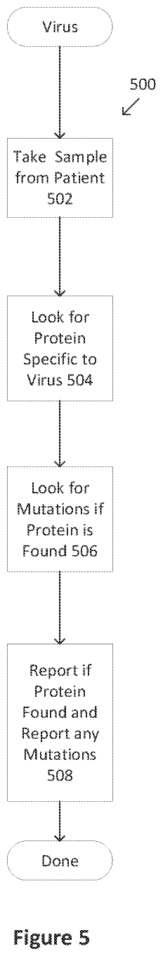

The approach of FIG. 1C has many possible applications. One application is to identify and track mutations of viruses. For example, with the SARS-CoV2 virus, one can track mutations of variants, such as the Delta variant and the Omicron variant. FIG. 5 depicts a flowchart 500 of illustrative steps that may be performed for such an application. At 502, a sample is obtained from a patient. This may be, for example, a nasal swab. If the patient has the virus, the sample will contain the protein that is found with the virus. For example, with the SARS-CoV2 virus, the n-capsid protein may be found. At 504, the sample is processed to isolate the protein. At 506, the protein is sequenced and processed as discussed above to locate and identify any mutations. If the protein is found in sufficient quantity, it is indicative of the patient having the virus. Any mutations in the protein may then be reported as described above.

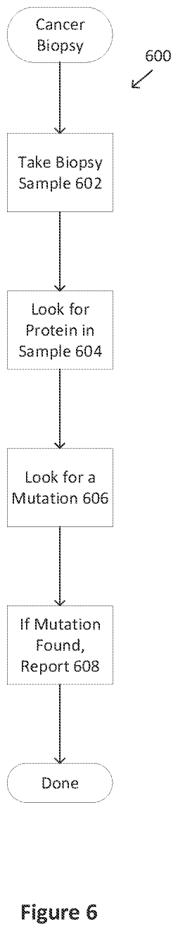

Another illustrative application is for testing patients for cancer. FIG. 6 depicts a flowchart of illustrative steps that may be performed to test whether a patient has cancer using the approach of FIG. 1C in exemplary embodiments. At 602, a biopsy sample is taken from the patient. The biopsy is taken from the site where the cancer is believed to be. The biopsy sample contains tissue that is to be tested to determine if the tissue contains cancer. At 604, the sample is processed to look for the presence of the protein in the sample. A particular mutation of the protein may be indicative of a cancer. If the protein is found in the sample, at 606, the protein is processed as described above to look for a specific mutation or set of mutations. If the mutation is found, at 608, the presence of the mutation is reported in the results of the test.

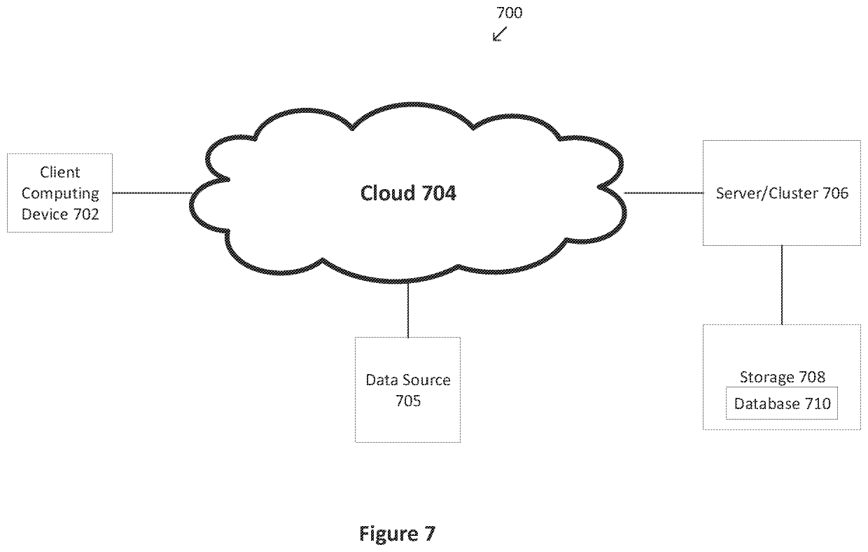

FIG. 7 depicts a computing environment that may be suitable for the exemplary embodiments. The functionality described relative to the processing of the sequences may be performed by a server computing device or a cluster 706, such as a cloud computing cluster. In such an arrangement, a request for processing sequences may originate from a client computing device 702. Alternatively, the processing of the sequences may occur on the client computing device 702 without requiring processing by the server/cluster 706. The client computing device 702 may be connected to the server/cluster via a network cloud 704 that may include the Internet, an intranet, a local area network and/or a wide area network, include computing networks as well as phone networks. The networks that are part of the network cloud 704 may include wired as well as wireless networks. A data source 705, such as a public database, may store the sequences that are imported by the server/cluster 706. The server/cluster 706 may have access to storage 708 that holds a database 710. The database 710 may be the “local database” referenced above. With the client/server arrangement or an application server arrangement, a client on the client computing device 702 requests that the server/cluster 706 process the sequences held in the database 710 and report the results back to the client computing device 702.

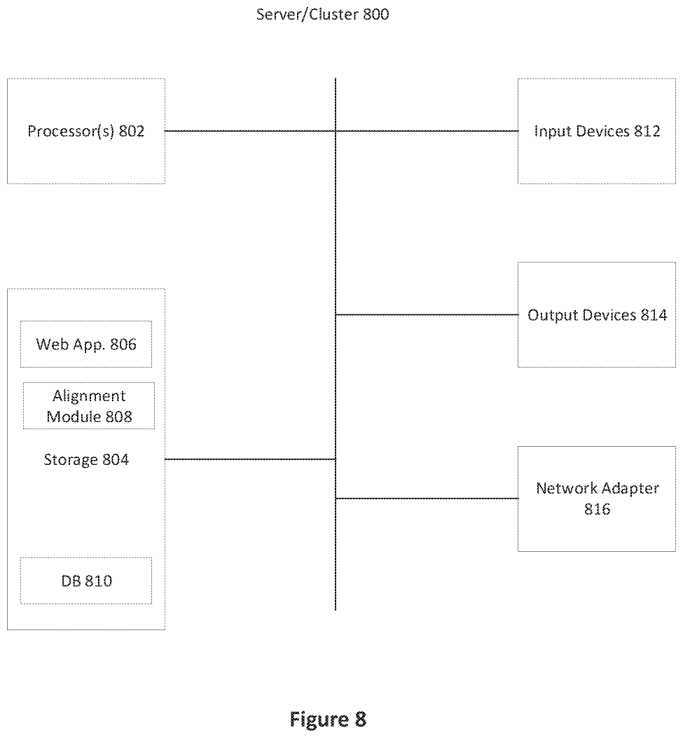

FIG. 8 depicts an illustrative depiction of the server/cluster 800 in exemplary embodiments. The server/cluster 800 may include one or more processors 802. The processors may be microprocessors, field programmable gate arrays (FPGAs), application specific integrated circuits (ASICs), graphics processing units (GPUs) or the like. The server/cluster 800 may include storage 804. The storage 804 may include a number of different types of non-transitory computer—readable storage media. For example, the storage 804 may include random access memory (RAM), read only memory (ROM), solid state memory, magnetic disk storage, optical disk storage and the like. The storage 804 may store a web application 806 for performing the processing of the sequences as discussed above. The storage 804 may also hold an alignment module 808 that aligns the sequences that are retrieved from a data source. The processor(s) may execute the web application 806 and the alignment module 806. The storage 804 may also store the local database 810. The server/cluster 800 may also include input devices 812, such as a mouse, a keyboard, a thumb pad, a microphone, etc. The server/cluster 800 may include output devices 814. The output devices 814 may include a display device, a printer and/or an audio output device. The server/cluster 800 may include a network adapter 816 for enabling the server/cluster 800 to have access to a network.

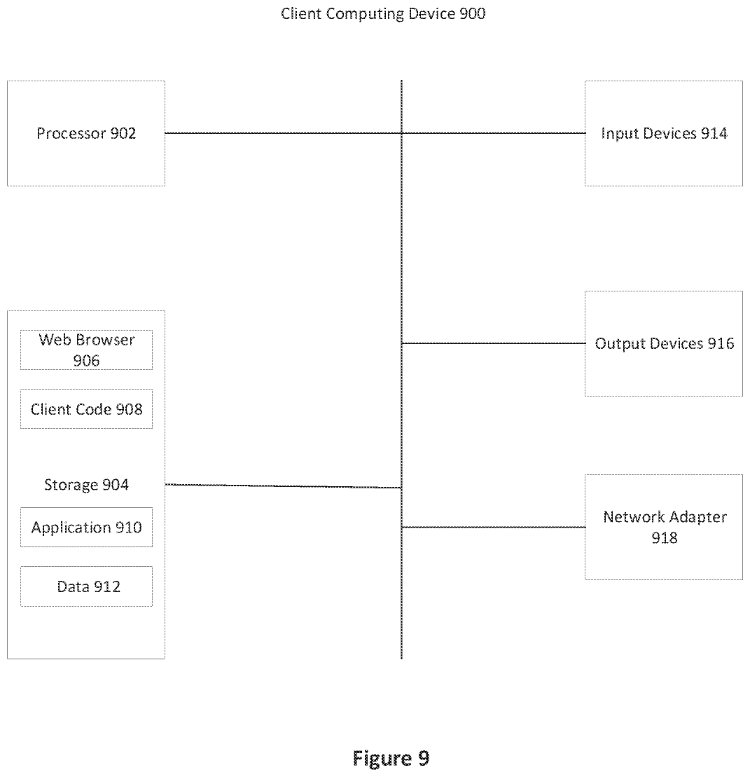

FIG. 9 depicts an example of a client computing device 900. The client computing device includes a processor 902, such as a microprocessor. The client computing device 900 includes storage 904. The storage 904 may include varieties of types of memory and storage such as detailed above for the storage 804 found in the server/cluster 800. The storage 904 may store a web browser 906 that may be executed by the processor 902 for the client gain access to web content, such as found on the Internet or an intranet. The storage 904 may also hold client code 908 for performing client operations when a client/server arrangement is used. The storage 904 may include an application 910. The application 910 may perform the functionality described above in receiving and processing sequences as described above. The storage 904 may also store data 912, including sequences and a local database. The client computing device 900 may include input devices 914, such as a mouse, a keyboard, a microphone, a thumb pad and the like. The client computing device 900 may include output devices 916, such as a display device, a printer, and audio output device, etc. the client computing device 900 may include a network adapter 918 for interfacing the client computing device 900 with the network cloud 504.

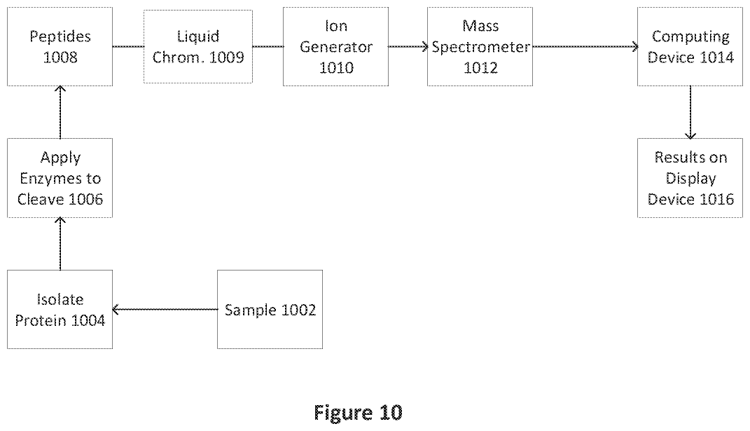

As was mentioned above, the exemplary embodiments may operate upon protein sequences. FIG. 10 depicts an illustrative approach for processing such protein sequences in exemplary embodiments. This approach includes not only the processing shown in FIG. 1C but also other operations detailed in FIG. 10. As was discussed above, a sample 1002 is obtained from a patient. The sample 1002 may take many different forms including, for example, that of a nasal swab, a blood sample, a urine sample or a saliva sample. The sample is processed to isolate a protein 1004. The protein may be isolated using known techniques. Enzymes may then be applied to cleave the protein into peptides 1006. This operation results in peptides 1008. These peptides 1008 may be passed into liquid chromatography system 1009 to separate the peptides. The output from the liquid chromatography system 1009 that includes the peptides 1008 may pass to an ion generator 1010. The ion generator 1010 ionizes the peptides 1008 and passes the peptides 1008 to a mass spectrometer 1012. The mass spectrometer 1012 is able to identify the ionized peptides by the indicative mass to charge ratios. It should be appreciated that is some embodiments a liquid chromatography or gas chromatography stage may be used before the ion generator 1010. The output from the mass spectrometer 1012 may be passed to a computing device 1014. The computing device 1014 may process was detected by the mass spectrometer 1012 to determine the partial sequence of the protein. The partial sequence may include the peptides of interest. This data may be further processed by the computing device 1014 as discussed above to identify any mutations and the frequency of such mutations over multiple sequences. The results are then displayed on a display device 1016.

While exemplary embodiments have been described herein, it should be appreciated that various changes in form and detail may be made relative to the exemplary embodiments without departing from the intended scope of the claims appended hereto.

1. A method performed by a processor of an electronic device, comprising:
programmatically determining with the processor that a selected subsequence for a protein, gene sequence, or nucleic acid is well suited for identifying mutations in gathered sequences of the protein or nucleic acid relative to a reference sequence;
analyzing the gathered sequences with the processor to identify any variations in the selected subsequence where the selected subsequence in the gathered sequences is different from the selected subsequence in the reference sequence;
for ones of the gathered sequences where one or more variations in the selected subsequence have been identified, determining with the processor whether the variation constitutes a mutation or not by applying a statistical test based on the frequency of the variation across the gathered sequences;
determining frequencies of mutations in the gathered sequences; and
outputting information regarding the frequencies of the mutations in the gathered sequences.

2. The method of claim 1, wherein the programmatically determining with the processor that a selected subsequence for a protein, gene sequence, or nucleic acid is well suited for identifying mutations in gathered sequences of the protein, the gene sequence, or the nucleic acid relative to a reference sequence, comprises:
designating subsequences that are specific to the protein, the gene sequence, or to the nucleic acid, subsequences in the protein, gene sequence, or nucleic acid that ionize well, and/or subsequences in the protein or nucleic acid that exhibit good mass selectivity as candidates for being the at least one selected subsequence in the protein, the gene sequence, or the nucleic acid that is well suited for identifying mutations in the gathered sequences relative to a reference sequence; and
choosing one or more subsequences among the candidates to be the at least one selected subsequence that is well suited for identifying mutations in the gathered sequences relative to the reference sequence.

3. The method of claim 1, wherein the programmatically determining with the processor that a selected subsequence for a protein, gene sequence, or nucleic acid is well suited for identifying mutations in gathered sequences of the protein, gene sequence or nucleic acid relative to a reference sequence determines that multiple subsequences are well suited for identifying mutations in the gathered sequences relative to the reference sequence.

4. The method of claim 1, further comprising programmatically with the processor retrieving the gathered sequences from a database.

5. The method of claim 1, further comprising sequence aligning the gathered sequences with the processor.

6. The method of claim 1, wherein the statistical test determines a likelihood for each variation and based on the likelihood, determines whether the variation is a mutation.

7. The method of claim 1, wherein the outputting information regarding the frequencies of the mutations in the gathered sequences comprises generating a web page, a file or a user interface element for display that contains the information regarding the frequencies of the mutations in the gathered sequences.

8. The method of claim 1, wherein the outputting information regarding the frequencies of the mutations in the gathered sequences comprises outputting graphics depicting the frequencies of the mutations in the gathered sequences.

9. The method of claim 8, wherein the outputting information regarding the frequencies of the mutations in the gathered sequences outputs the frequencies sorted by location where the gathered sequences were gathered and/or the dates when the gathered sequences were gathered.

10. The method of claim 1, wherein the outputting information regarding the frequencies of the mutations in the gathered sequences comprises outputting what each of mutations is and a frequency of each of the mutations.

11. The method of claim 1, wherein the protein is one of a protein found in a virus, disease or disorder.

12. A non-transitory processor-readable storage medium for computer programming instructions for execution by a processor to cause the processor to:
programmatically determine that a selected subsequence is well suited for identifying mutations in gathered sequences of a protein, gene sequence, or a nucleic acid relative to a reference sequence of the protein, gene sequence or nucleic acid;
analyze the gathered sequences to identify variations, wherein the selected subsequence in the gathered sequences is different from the at least one subsequence in the reference sequence;
for ones of the gathered sequences where the selected subsequence is identified as different from the one subsequence in the reference sequence, determine whether there is a mutation or a non-mutation variation by applying a statistical test;
determine frequencies of mutations in the gathered sequences; and
output information regarding the frequencies of the mutations in the gathered sequences.

13. The non-transitory processor-readable storage medium of claim 12, wherein the gathered sequences are one of protein sequences, gene sequences, DNA sequences or RNA sequences.

14. The non-transitory processor-readable storage medium of claim 12, wherein the computer programming instructions for execution by a processor to cause the processor to programmatically determine that selected subsequence is well suited for identifying mutations in gathered sequences of a protein, gene sequence, or a nucleic acid relative to a reference sequence of a protein or nucleic acid, comprises computer programming instructions that cause the processor to:
designate subsequences that are specific to the reference sequence, subsequences in the gathered sequences that ionize well, and/or subsequences that exhibit good mass selectivity as candidates for being the selected subsequence that is well suited for identifying mutations in the gathered sequences relative to the reference sequence; and
choosing a subsequence among the candidates to be the selected subsequence that is well suited for identifying mutations in the gathered sequences relative to the reference sequence.

15. The non-transitory processor-readable storage medium of claim 12, wherein the programmatically determining that the selected subsequence in the gathered sequences is well suited for identifying mutations in the gathered sequences relative to a reference sequence determines that multiple subsequences are well suited for identifying mutations in the gathered sequences relative to the reference sequence.

16. The non-transitory processor-readable storage medium of claim 12, further storing computer programming instructions that when executed by the processor cause the processor to sequence align the gathered sequences.

17. The non-transitory processor-readable storage medium of claim 12, wherein the statistical test determines for each variation a likelihood of the variation is and based on the likelihood, determines whether the variation is a mutation.

18. The non-transitory processor-readable storage medium of claim 12, wherein the outputting information regarding the frequencies of the mutations in the gathered sequences outputs the frequencies sorted by location where the gathered sequences were gathered and/or the dates when the gathered sequences were gathered.

19. An electronic device, comprising:
a storage for storing computer programming instructions; and
a processor configured to execute the computer programming instructions to:
programmatically determine that a selected subsequence is well suited for identifying mutations in the gathered sequences of a protein, gene sequence or nucleic acid relative to a reference sequence;
analyze the gathered sequences to identify variations where the selected subsequence in the gathered sequences is different from the selected subsequence in the reference sequence;
for ones of the gathered sequences where the selected subsequence is identified as different from the subsequence in the reference sequence, determine whether there is a mutation or a non-mutation variation by applying a statistical test;
determine frequencies of mutations in the gathered sequences; and
output information regarding the frequencies of the mutations in the gathered sequences.

20. The electronic device of claim 19, wherein the outputting information regarding the frequencies of the mutations in the gathered sequences comprises generating a web page, a file or a user interface element for display that contains the information regarding the frequencies of the mutations in the gathered sequences.
